(19) United States
(12) Patent Application Publication
(10) Pub. No.: US 2013/0296576 A1
(43) Pub. Date: Nov. 7, 2013

(54) HIGH STABLE NON-IONIC N-VINYL BUTYROLACTAM IODINE AND PREPARATION METHOD THEREOF

(71) Applicants: Yu Wang, Shanghai (CN); Zhan Chen, Shanghai (CN)

(72) Inventors: Yu Wang, Shanghai (CN); Zhan Chen, Shanghai (CN)

(73) Assignee: SHANGHAI YUKING WATER SOLUBLE MATERIAL TECH CO., LTD., Shanghai (CN)

(21) Appl. No.: 13/978,329

(22) PCT Filed: Aug. 18, 2011
(86) PCT No.: PCT/CN2011/078573

§ 371 Date: Jul. 3, 2013

(30) Foreign Application Priority Data
Jan. 7, 2011 (CN) 201110002653.X

(51) Int. Cl. C07D 207/267

(52) U.S. Cl. CPC C07D 207/267
USPC 548/543

(57) ABSTRACT

The present invention relates to a preparation method of a high-stable non-ionic N-vinyl butyrolactam iodine, wherein non-ionic N-vinyl butyrolactam, iodine and at least one grinding aid are stirred at 150-800 r/min at a temperature of 50° C.-90° C. for 1 to 12 hours to prepare the high-stable non-ionic N-vinyl butyrolactam iodine, wherein the K value of the non-ionic N-vinyl butyrolactam is 32±1, the PD value of the main peak of the non-ionic N-vinyl butyrolactam is ≦1.6, the moisture content of the non-ionic N-vinyl butyrolactam is ≦2.5%, preferably, the grinding aid is selected one or several from sodium chloride, sodium citrate, sodium carbonate and sodium phosphate, the amount of the grinding aid added is 0.02 to 2% of the total amount of the non-ionic N-vinyl butyrolactam and the iodine, the non-ionic N-vinyl butyrolactam is PVP-K32, the high-stable non-ionic N-vinyl butyrolactam iodine prepared by the above mentioned method is also provided, the stability of the high-stable non-ionic N-vinyl butyrolactam iodine of the present invention is high, thereby facilitating long-term storage and use, thus the high-stable non-ionic N-vinyl butyrolactam iodine is suitable for large-scale popularization.

### FIELD OF TECHNOLOGY

The present invention relates to the technical field of compounds, especially to the technical field of non-ionic N-vinyl butyrolactam iodines, in particular to a high-stable non-ionic N-vinyl butyrolactam iodine and preparation method thereof.

### DESCRIPTION OF RELATED ARTS

Non-ionic N-vinyl butyrolactam iodine (PVP-I) is a kind of amorphous powders having yellowish-brown to reddish-brown, an unshaped iodine complex formed by complexing non-ionic N-vinyl butyrolactam (PVP) with iodine, and an indophor. PVP-I is an excellent disinfectant, and collected in Chinese Pharmacopoeia of 1990 edition. It can maintain a bactericidal power for a longer time, is a broad-spectrum strong disinfectant, has a relatively strong role in killing viruses, bacteria, fungi and mold spores, and is approved by the Chinese Pharmacopoeia as a sanitizer that can be used directly on human bodies.

Non-ionic N-vinyl butyrolactam (PVP) is a non-ionic surfactant, itself has no antibacterial effect, but can increase the solubility of iodine, helps to improve the wetting and penetration abilities of an iodine solution to an object, such as to enhance the affinity of the available iodine to cell membranes, so that the available iodine can be introduced directly into the cell membranes and the cytoplasms of bacteria, and kill the bacteria in a few seconds immediately, thereby enhancing the bactericidal ability of iodine, and PVP-I also is easy to be dissolved in water, has no irritation, allergy and poisoning to skins and mucous membranes, thus it is evaluated very good domestic and abroad. PVP-I not only can be used as an aqueous solution, but also can be used in the solid form in some special occasions. This makes the PVP-I complex be widely used in sterilization, disinfection of various fields, expands the application range of iodine, and makes the PVP-I complex be widely applied in the disinfection of the domestic hospitals.

But the stability of the PVP-I produced by traditional methods is not ideal, during storage, due to easy decomposition and sublimation, the content of its available iodine is decreased slightly, and can not meet the quality standard we required, and it is more unstable especially in summer, to bring about inconvenience to long-term storage and use.

Therefore, there is an urgent need to provide a high-stable non-ionic N-vinyl butyrolactam iodine, whose stability is high, thereby facilitating long-term storage and use.

### SUMMARY OF THE INVENTION

Aspects of the present invention generally pertain to a high-stable non-ionic N-vinyl butyrolactam iodine and preparation method thereof, the stability of the high-stable non-ionic N-vinyl butyrolactam iodine is high, thereby facilitating long-term storage and use, thus the high-stable non-ionic N-vinyl butyrolactam iodine is suitable for large-scale popularization.

In order to realize the above aims, in a first aspect of the present invention, a preparation method of a high-stable non-ionic N-vinyl butyrolactam iodine is provided and characterized in that, non-ionic N-vinyl butyrolactam, iodine and at least one grinding aid are stirred at 150-800 r/min at a temperature of 50° C.-90° C. for 1 to 12 hours to prepare the high-stable non-ionic N-vinyl butyrolactam iodine, wherein the K value of the non-ionic N-vinyl butyrolactam is 32±1, the PD value of the main peak of the non-ionic N-vinyl butyrolactam is ≦1.6, the moisture content of the non-ionic N-vinyl butyrolactam is ≦2.5%. Wherein the iodine can be iodine plates or iodine powders, preferably, the iodine is adopted as iodine powders.

The above mentioned reaction time is determined by recording the starting reaction time, sampling one time every half an hour, measuring the available iodine content and stopping the reaction when the reaction is performed about 4 h and the available iodine content of the product reaches up to about 11.0%, therefore, if a higher available iodine content is needed, the reaction time may be extended appropriately.

The grinding aid can adopt any suitable grinding aid. Preferably, the grinding aid is selected one or several from sodium chloride, sodium citrate, sodium carbonate and sodium phosphate.

The person skilled in the art can add a suitable amount of the grinding aid according to the experience. Preferably, the amount of the grinding aid added is 0.02 to 2% of the total amount of the non-ionic N-vinyl butyrolactam and the iodine.

More preferably, the non-ionic N-vinyl butyrolactam is 42 g, the iodine is 8 g and the grinding aid is 0.01-1.0 g.

The temperature can be provided in any suitable manner. Preferably, the temperature is provided in an oil bath manner.

The non-ionic N-vinyl butyrolactam can be provided in any suitable manner. Preferably, the non-ionic N-vinyl butyrolactam is obtained by reacting N-vinyl butyrolactam (NVP), an initiator, and water at 55-80° C. in an inert gas for 0.5 to 8 hours and then drying the product. The drying can adopt a vacuum oven for example. The initiator can adopt azobisisobutyronitrile for example.

The inert gas can be any suitable gas. More preferably, the inert gas is nitrogen.

The non-ionic N-vinyl butyrolactam can be any non-ionic N-vinyl butyrolactam meeting the

K value 32±1, the PD value of the main peak ≦1.6, the moisture content ≦2.5%. Preferably, the non-ionic N-vinyl butyrolactam is PVP-K32.

In a second aspect of the present invention, a high-stable non-ionic N-vinyl butyrolactam iodine is provided and characterized in that, the high-stable non-ionic N-vinyl butyrolactam iodine is obtained by the preparation method of a high-stable non-ionic N-vinyl butyrolactam iodine mentioned above.

The beneficial effects of the present invention are as follows:

1. The present invention prepares the high-stable non-ionic N-vinyl butyrolactam iodine by stirring non-ionic N-vinyl butyrolactam, iodine and at least one grinding aid at 150-800 r/min at a temperature of 50° C. -90° C. for 1 to 12 hours, wherein the K value of the non-ionic N-vinyl butyrolactam is 32±1, the PD value of the main peak of the non-ionic N-vinyl butyrolactam is ≦1.6, the moisture content of the non-ionic N-vinyl butyrolactam is ≦2.5%, therefore the stability of the high-stable non-ionic N-vinyl butyrolactam iodine prepared is high, thereby facilitating long-term storage and use, thus the high-stable non-ionic N-vinyl butyrolactam iodine is suitable for large-scale popularization.

2. The present invention does not use the conventional PVPK30, instead use non-ionic N-vinyl butyrolactam with the K value 32±1, the PD value of the main peak ≦1.6, and the moisture content ≦2.5%, such as the PVP-K32 product manufactured by Shanghai Yu King Chemical Technology Development Co., Ltd., compared with the conventional PVPK30, the K value of PVP-K32 is relatively higher, the PD value characterizing the molecular weight distribution of PVP-K32 is relatively smaller, the moisture content of PVP-K32 is also relatively smaller; the combined effect of the three values guarantees that the reaction proceeds completely. The reason is that: PVP with the higher K value has a relatively long molecular chain, and the active points are relatively more correspondingly, and the narrower molecular weight distribution shows that the polymers distribute uniformly and the distribution of the active points of the molecular chain is regular. Meanwhile the smaller moisture content enhances the flowability of PVPK32, so as to be able to increase the probability of reacting it with the iodine during the same time. The synergistic effect of the three values makes PVP and the iodine complex completely and sufficiently, and increases the stability of the product.

### DETAILED DESCRIPTION OF THE PREFERRED EMBODIMENT

In order to understand the technical content of the present invention clearly, the present invention is further exemplified by reference to the following examples. The raw materials and equipments involved in the examples are as follows:

Experimental materials: PVP-K32 (pharmaceutical grade, the K value 32±1, the PD value of the main peak ≦1.6; the moisture content ≦2.5%, Shanghai Yu King Chemical Technology Development Co., Ltd.), iodine (99.5%, pharmaceutical grade, Turkmenistan), a starch indicator (made by the applicant), a sodium thiosulfate standard solution (made by the applicant), sodium citrate (reagent grade).

Instruments and equipments:

| Instrument name | Model | Place of origin |
|---|---|---|
| high-speed crushing | DFT-100 | Shanghai Dingguang Machinery |
| machine | | Co., Ltd. |
| Air blowing thermostatic | DZF-6021 | Shanghai Suopu Instrument |
| drying oven | | Co., Ltd. |
| Constant speed stirrer | S312 | Shanghai Meiyingpu Instrument |
| | | Manufacturing Co., Ltd. |
| Electronic analytical | FA2004 | Shanghai Liangping Instrument |
| balance | | Co., Ltd. |
| Thermostatic water bath | RE-205 | Gongyi Yingyuyuhua Instrument |
| | | Plant |
| Alkali burette | 50 ml | Shanghai Heqi Glass Instrument |
| | | Co., Ltd. |

The related test methods used are as follows:

Available iodine test: A sample concentration can be determined by reference to related methods of the Chinese Pharmacopoeia. The present experiment measures the available iodine content according to USP (United States Pharmacopoeia): a sample of about 1 g is weighed accurately and then put into a beaker, dissolved completely by adding appropriate water and stirring, then transferred into a 100 ml volumetric flask, 2 copies of 20 ml sample solution are pipetted precisely to an iodine flask respectively, and each is titrated with a sodium thiosulfate standard titration solution (0.1 mol/L), when the color is near light yellow, an appropriate amount of a starch indicator is added by dropping, and the sodium thiosulfate drops continuously till the blue color disappears, the volume of the sodium thiosulfate used is recorded and the available iodine content of the sample is calculated.

Stability test: The acceleration experiment is used, a certain amount of a sample is weighed accurately and prepared to a 1% solution, an appropriate amount of which is pipetted into an iodine flask, placed at 85° C. for 15 h, then its available iodine content is measured, and the reduced ratio of the available iodine is calculated.

### Embodiment 1

8.0 g crushed iodine powders and 42.0 g PVP-K30 are weighed accurately, added into a 250 ml three-necked flask at the same time, and react for about 4 h in an oil bath temperature of 80° C. and at a speed of 150 r/min provided by a stirrer.

### Embodiment 2

8.0 g crushed iodine powders and 42.0 g PVP-K32 are weighed accurately, added into a 250 ml three-necked flask at the same time, and react for about 4 h in an oil bath temperature of 80° C. and at a speed of 150 r/min provided by a stirrer.

### Embodiment 3

8.0 g crushed iodine powders, 42.0 g PVP-K30 and 0.5 g sodium citrate are weighed accurately, added into a 250 ml three-necked flask at the same time, and react for about 4 h in an oil bath temperature of 80° C. and at a speed of 800 r/min provided by a stirrer.

### Embodiment 4

8.0 g crushed iodine powders, 42.0 g PVP-K32 and 0.5 g sodium citrate are weighed accurately, added into a 250 ml three-necked flask at the same time, and react for about 4 h in an oil bath temperature of 80° C. and at a speed of 800 r/min provided by a stirrer.

### Embodiment 5

8.0 g crushed iodine powders, 42.0 g PVP-K32 and 0.01 g sodium citrate, 0.5 g sodium carbonate are weighed accurately, added into a 250 ml three-necked flask at the same time, and react for about 1 h in an oil bath temperature of 50° C. and at a speed of 400 r/min provided by a stirrer.

### Embodiment 6

8.0 g crushed iodine powders, 42.0 g PVP-K32 and 1.0 g sodium citrate are weighed accurately, added into a 250 ml three-necked flask at the same time, and react for about 12 h in an oil bath temperature of 90° C. and at a speed of 800 r/min provided by a stirrer.

The results of the available iodine tests and the stability tests of PVPIs prepared in Embodiments 1-6 mentioned are as follows:

TABLE 1
the available iodine contents and the stabilities of 1% PVPI solutions
stored at 85° C. for 15 h

| | Initial available | available iodine | changed value of |
|---|---|---|---|
| | iodine | content | available |
| Number | content (%) | (%) after 15 h | iodine (%) |
| Embodiment 1 | 11.15 | 8.59 | −22.96 |
| Embodiment 2 | 11.21 | 10.03 | −10.52 |
| Embodiment 3 | 11.27 | 10.65 | −5.47 |
| Embodiment 4 | 11.32 | 10.96 | −3.11 |
| Embodiment 5 | 11.19 | 10.82 | −3.22 |
| Embodiment 6 | 11.22 | 10.86 | −3.17 |

Note:
The available iodine content is calculated based on the dry product.

The stability data results from the above table show that the stabilities of the PVPI samples prepared by adding the grinding aid sodium citrate are much higher than the ordinary PVPI powders prepared without adding the grinding aid. The reasons are that: firstly, the sodium citrate added plays the role of grinding aids, promotes the contact of the iodine and PVP-K32 to strengthen the internal friction, so as to make the complexing complete and the PVPIs obtained more stable; secondly, compared the high active PVP-K32 product manufactured by Shanghai Yu King Chemical Technology Development Co., Ltd. with the PVPK30 manufactured by other manufacturers, the K value of PVP-K32 is relatively higher, the PD value characterizing the molecular weight distribution of PVP-K32 is relatively smaller, the moisture content of PVP-K32 is also relatively smaller; the combined effect of the three values guarantees that the reaction proceeds completely. The reason is that: PVP with the higher K value has a relatively long molecular chain, and the active points are relatively more correspondingly, and the narrower molecular weight distribution shows that the polymers distribute uniformly and the distribution of the active points of the molecular chain is regular. Meanwhile the smaller moisture content enhances the flowability of PVPK32, so as to be able to increase the probability of reacting it with the iodine during the same time. The synergistic effect of the three values makes PVP and the iodine complex completely and sufficiently, and increases the stability of the product.

Therefore, the use of the raw material PVP with a molecular weight of K32, the PD value less than 1.6, the moisture content less than 2.5, is a key factor in preparing the stable PVPI, the grinding aid may be selected one or several from sodium chloride, sodium citrate, sodium carbonate and sodium phosphate. The available iodine content of the PVPI powders prepared by the present method can reach up to 11% or more, the stability is increased greatly, and the shelf life is over 3 years. Thus the PVPI powders can fully meet the market requirements. Moreover, the storage time of the solutions prepared with the PVPI samples are extended greatly, and are conducive to the long-term storage of PVPI disinfectants.

To sum up, the stability of the high-stable non-ionic N-vinyl butyrolactam iodine of the present invention is high, thereby facilitating long-term storage and use, thus the high-stable non-ionic N-vinyl butyrolactam iodine is suitable for large-scale popularization.

In the present specification, the present invention has been described according to the particular embodiments. But it is obvious that these embodiments can be modified or changed without departure from the spirit and scope of the present invention. Therefore, the specification described above is exemplary only and not intended to be limiting.

1. A preparation method of a high-stable non-ionic N-vinyl butyrolactam iodine, characterized in that, non-ionic N-vinyl butyrolactam, iodine and at least one grinding aid are stirred at 150-800 r/min at a temperature of 50° C.-90° C. for 1 to 12 hours to prepare the high-stable non-ionic N-vinyl butyrolactam iodine, wherein the K value of the non-ionic N-vinyl butyrolactam is 32±1, the PD value of the main peak of the non-ionic N-vinyl butyrolactam is ≦1.6, the moisture content of the non-ionic N-vinyl butyrolactam is ≦2.5%.

2. The preparation method of a high-stable non-ionic N-vinyl butyrolactam iodine according to claim 1, characterized in that, the grinding aid is selected one or several from sodium chloride, sodium citrate, sodium carbonate and sodium phosphate.

3. The preparation method of a high-stable non-ionic N-vinyl butyrolactam iodine according to claim 1, characterized in that, the amount of the grinding aid added is 0.02 to 2% of the total amount of the non-ionic N-vinyl butyrolactam and the iodine.

4. The preparation method of a high-stable non-ionic N-vinyl butyrolactam iodine according to claim 3, characterized in that, the non-ionic N-vinyl butyrolactam is 42 g, the iodine is 8 g and the grinding aid is 0.01-1.0 g.

5. The preparation method of a high-stable non-ionic N-vinyl butyrolactam iodine according to claim 1, characterized in that, the temperature is provided in an oil bath manner.

6. The preparation method of a high-stable non-ionic N-vinyl butyrolactam iodine according to claim 1, characterized in that, the non-ionic N-vinyl butyrolactam is obtained by reacting N-vinyl butyrolactam (NVP), an initiator, and water at 55-80° C. in an inert gas for 0.5 to 8 hours and then drying the product.

7. The preparation method of a high-stable non-ionic N-vinyl butyrolactam iodine according to claim 6, characterized in that, the inert gas is nitrogen.

8. The preparation method of a high-stable non-ionic N-vinyl butyrolactam iodine according to claim 1, characterized in that, the non-ionic N-vinyl butyrolactam is PVP-K32.

9. A high-stable non-ionic N-vinyl butyrolactam iodine, characterized in that, the high-stable non-ionic N-vinyl butyrolactam iodine is obtained by the preparation method of a high-stable non-ionic N-vinyl butyrolactam iodine according to claim 1.
